(19) United States
(12) Patent Application Publication
(10) Pub. No.: US 2022/0048994 A1
(43) Pub. Date: Feb. 17, 2022

(54) TRIFUNCTIONAL ANTIGEN-BINDING MOLECULE

(71) Applicants: Melvyn Little, St. Peter-Ording (DE); Eugene Zhukovsky, Mannheim (DE); Markus Eser, Düsseldorf (DE); Michael Weichel, Bischofsheim (DE); Thorsten Gantke, Edingen-Neckarhausen (DE); Uwe Reusch, Maikammer (DE); Kristina Ellwanger, Heidelberg (DE); Fabrice Le Gall, Mainz (DE)

(72) Inventors: Melvyn Little, St. Peter-Ording (DE); Eugene Zhukovsky, Mannheim (DE); Markus Eser, Düsseldorf (DE); Michael Weichel, Bischofsheim (DE); Thorsten Gantke, Edingen-Neckarhausen (DE); Uwe Reusch, Maikammer (DE); Kristina Ellwanger, Heidelberg (DE); Fabrice Le Gall, Mainz (DE)

(21) Appl. No.: 17/517,869

(22) Filed: Nov. 3, 2021

Related U.S. Application Data
(63) Continuation of application No. 15/290,255, filed on Oct. 11, 2016, now abandoned.
Application No. 15/290,255 is a continuation-in-part of application No. PCT/EP2015/057919, filed on Apr. 12, 2015.

(30) Foreign Application Priority Data
Apr. 13, 2014 (EP) 14164523.4

(51) Int. Cl. C07K 16/28; C07K 16/46

(52) U.S. Cl. CPC C07K 16/2803; C07K 16/2809; C07K 16/2878; C07K 16/468; C07K 2317/73; C07K 2317/622; C07K 2317/626; C07K 2317/35; C07K 2317/14; C07K 2317/31

(57) ABSTRACT

The invention relates to a trispecific antigen-binding molecule, wherein the antigen-binding molecule is at least tetravalent and comprises an antigen-binding site having specificity against a first antigen epitope, an antigen-binding site having specificity against a second antigen epitope and two antigen-binding sites having specificity against a third antigen epitope and its use a medicament for tumor therapy.

### RELATED APPLICATIONS AND INCORPORATION BY REFERENCE

This application is a continuation of U.S. application Ser. No. 15/290,255 filed Oct. 11, 2016, which is a continuation-in-part application of international patent application no. PCT/EP2015/057919 filed Apr. 12, 2015, which published as PCT Publication No. WO 2015/158636 on Oct. 22, 2015, which claims benefit of European patent application Serial No. EP 14164523.4 filed Apr. 13, 2014.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been submitted electronically in ASCII format and is hereby incorporated by reference in its entirety. Said ASCII copy, is named Y8004_00003SL.txt and is 17.5 bytes in size.

### FIELD OF THE INVENTION

The present invention relates to a multifunctional, for example trifunctional, antigen-binding molecule and its therapeutic application, for example in immunotherapy. The molecule is a Fv-antibody derivative. In certain embodiments the invention relates to multimeric, for example dimeric, antigen binding molecules.

### BACKGROUND OF THE INVENTION

Bispecific, i.e. bifunctional, antibodies can be used to engage two different therapeutic targets or perform two distinct functions. Such antibodies can be used for example to recruit an immune effector cell, e.g. T- or NK-cell, towards a particular target cell. Various antibody-fragment based molecules are known and under investigation, for example for cancer therapy.

Bifunctional and dimeric antibodies can be constructed using only antibody variable domains. For example, the linker sequence between the VH and VL domains can be shortened to such an extent that they cannot fold over and bind one another in an intramolecular fashion. Such short linkers, e.g. 2-12 residues, prevent said folding of a scFv molecule and favor intermolecular VH-VL pairings between complementary variable domains of different polypeptide chains forming a dimeric “diabody” (Holliger et al., 1993, Proc. Natl. Acad. Sci. USA 90, 6444-6448). Such diabody can be used for bifunctional antibodies, which are obtained by non-covalent association of two single-chain polypeptide fusion products, each consisting of the VH domain from one antibody connected by a short linker to the VL domain of another antibody.

WO 03/025018 discloses a bispecific and multimeric antigen-binding molecule which structure is formed by identical single-chain polypeptides with at least four binding domains. A VH and a VL domain at a terminal part of each polypeptide chain are linked by a short linker and associate intermolecularly with the corresponding VH and VL domains of another polypeptide chain, while the other VH and VL domains of each polypeptide chain bind intramolecularly to one another within the same chain resulting in an antigen-binding scFv unit. Such constructs are homodimers, i.e. they consist of identical single-chain polypeptides associated with one another.

Citation or identification of any document in this application is not an admission that such document is available as prior art to the present invention.

### SUMMARY OF THE INVENTION

Provided herein are multifunctional antigen-binding molecules, which are at least trifunctional. In some embodiments the trifunctional antigen-binding molecule is at least trispecific, i.e. has specificity for at least three different antigen epitopes.

The antigen-binding molecule according to the invention is a Fv-derivative which may comprise only variable (Fv) antibody domains, but is devoid of constant antibody domains. The variable (Fv) antibody domains of the antigen-binding molecule are linked with one another by a peptide linker or a peptide bond. The antigen-binding molecule according to the invention can be a monomer of a single polypeptide chain or a multimer of a multichain polypeptide. A multimeric antigen-binding molecule can be, for example, a dimer having two polypeptide chains, a trimer having three polypeptide chains or a tetramer having four polypeptide chains.

In some embodiments the trispecific antigen-binding molecule is at least tetravalent. The trispecific and tetravalent antigen-binding molecule may comprise an antigen-binding site having specificity against a first antigen epitope, an antigen-binding site having specificity against a second antigen epitope and two antigen-binding sites having specificity against a third antigen epitope. Thus, this trispecific and tetravalent antigen-binding molecule has different specificities for three different antigen epitopes. For example, such antigen-binding molecule may comprise a first antigen-binding site having specificity against a first antigen epitope, a second antigen-binding site having specificity against a second antigen epitope, a third and a fourth antigen-binding sites having specificity against a third antigen epitope. In some embodiments where the trispecific and tetravalent antigen-binding molecule is a multimer, the antigen-binding molecule is heterodimeric, i.e. may comprise at least two different polypeptide chains, wherein these two polypeptide chains differ in at least one variable domain, e.g. one polypeptide chain may comprise only a VH domain and the other one may comprise only the respective VL domain of the same antigen epitope specificity.

Because the tetravalent antigen-binding molecule may comprise eight antibody variable domains its molecular weight is above 100 kDa which results in a longer half-life of such a molecule compared with trivalent and trispecific single-chain Fv molecules.

Further, each trispecific and tetravalent antigen-binding molecule may comprise two antigen-binding sites having specificity for the same antigen epitope. Thereby the avidity is increased, i.e. the strength of interaction between the antigen epitope and antigen-binding molecule. Advantages of the higher avidity are increased stability of interaction and retention on the target. For example, if the target is a cytotoxic immune effector cell such as a T-cell or a NK-cell, the higher avidity can result in an increased cytolytic potential of the antigen-binding molecule. In another example, if the target is a tumor cell, the higher avidity improves the retention time on the target and reduces the off-rates from the target. In a certain embodiment of the invention, the trispecific and tetravalent antigen-binding molecule may comprise a first and a second antigen-binding sites specific for two different antigen epitopes of the same kind of tumor cell and a third and a fourth antigen binding sites specific for an antigen epitope on an immune effector cell, such as T-cell or NK-cell. Such an antigen-binding molecule leads to an increased specificity as well as avidity for a particular kind of tumor cell and to an increased avidity for activating a receptor on the immune effector cell which results in an advantageously increased specific cytolytic potential of the antigen-binding molecule. The binding to two distinct tumor antigen epitopes leads to an increase in targeting specificity and to an extension of the therapeutic window by reducing off-target toxicities. Importantly, despite the structural complexity, such trispecific and tetravalent antigen-binding molecule according to the invention is stable.

Therefore, the antigen-binding molecule according to the invention can be utilized in different ways for redirecting the cytotoxic potential of immune effector cells to destroy tumor cells or infectious agents. In some embodiments the trispecific antigen-binding molecule may bind to two different antigen epitopes on a target. For example, the two different epitopes may be on the same antigen to prevent escape mutants or to enhance efficacy or the two epitopes may be on two different antigens of the target. In other embodiments the trispecific antigen-binding molecule may bind to two different antigen epitopes on immune effector cells. For example, a first antigen-binding site has specificity for an activating receptor, e.g. CD16A or CD3, and a second antigen-binding site has specificity for a co-stimulatory receptor, e.g., CD137 or CD28. In another example, a first antigen-binding site has specificity for CD16A and a second antigen-binding site for another activating receptor on NK cells, e.g. NKG2D, DNAM, NCRs).

In another embodiment the trispecific antigen-binding molecule has a first antigen-binding site having specificity for an antigen epitope on a tumor cell, a second antigen-binding site having specificity for an antigen epitope on an immune effector cell and a third antigen-binding site having specificity for an antigen epitope on a soluble protein selected from the group of growth factors, cytokines, chemokines, mitogens and albumins. Examples of such a soluble protein are IL-6, BAFF, APRIL, TGF-beta, IL-10, VEGF-A, HB-EGF, angiopoietin-2 and human serum albumin (HSA).

In an alternative embodiment the antigen-binding molecule has one antigen-binding site having specificity for an antigen epitope of an antigen present on one type of cell and three antigen-binding sites having specificities of antigen epitopes on one or more other types of cells.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved Nothing herein is to be construed as a promise.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

The following detailed description, given by way of example, but not intended to limit the invention solely to the specific embodiments described, may best be understood in conjunction with the accompanying drawings.

FIG. 1 shows a first and a second polypeptide for forming a trifunctional, i.e. trispecific, antigen-binding polypeptide dimer according to the invention. The first polypeptide has four antibody variable domains VH, VL, VH, VH linked one after another. The first and the second VH antibody variable domains (black) have the same first specificity and are linked by a short linker L3 for preventing intramolecular pairing within the same polypeptide and a single-chain Fv unit having an antibody variable domain pair of the other third variable antibody domain VL and the fourth variable antibody domain VH (white) linked by a second linker L1 capable of intramolecularly forming an antigen binding site of a second specificity by the variable domain pair within the same polypeptide. The second antibody variable domain VH and the third antibody variable domain VL of different specificities are linked by a third linker L2.

The second polypeptide has four antibody variable domains VL, VL, VL, VH linked one after another. The first and the second VL antibody variable domains (black) have the same first specificity and are linked by a short linker L4 for preventing intramolecular pairing within the same polypeptide and a single-chain Fv unit having an antibody variable domain pair of the other third variable antibody domain VL and the fourth variable antibody domain VH having a third specificity (grey) and are linked by a second linker L1 capable of intramolecularly forming an antigen binding site by the variable domain pair within the same polypeptide. The second antibody variable domain VL and the third antibody variable domain VL of different specificities are linked by a third linker L2.

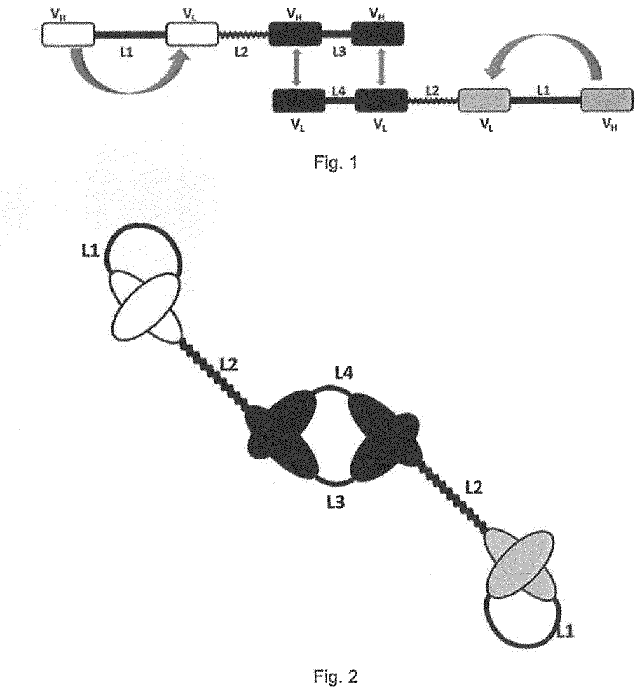

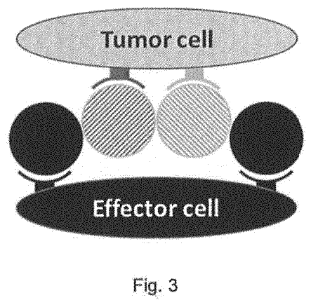

FIG. 2 shows the antigen-binding polypeptide dimer formed by non-covalent association between the two polypeptides of FIG. 1, whereas the two antibody variable VH domains linked by a short linker of the first polypeptide associated with the two corresponding antibody variable VL domains of the second polypeptide, thereby forming two antigen binding sites having the same specificity (black), whereas the second specificity is provided by the single chain Fv unit of the first polypeptide (white) and the third specificity is provided by the single chain Fv unit of the second polypeptide (grey).

FIG. 3 shows a trifunctional antigen-binding molecule, in particular trifunctional antigen-binding polypeptide, according to the invention which is a trispecific antibody for dual targeting of tumor cells. The antibody, i.e. antigen-binding polypeptide, is designed to target two different targets/epitopes on the tumor cell and with the third functionality bind with high affinity to an effector cell. The antigen-binding polypeptide consists of four antigen binding sites, wherein the two central antigen binding sites bind to two different antigens on the tumor cell and the two peripheral antigen binding sites bind to the effector cell.

### DETAILED DESCRIPTION OF THE INVENTION

“Tetravalent” means that the antigen-binding molecule may comprise four antigen-binding sites, wherein each of the antigen-binding sites may comprise a VH/VL pair having a variable heavy chain (VH) domain and a variable light chain (VL) domain of the same antigen epitope specificity associated with one another. Thus, such tetravalent antigen-binding molecule may comprise at least eight variable antibody domains, namely four variable heavy chain (VH) domains and four variable light chain (VL) domains. “Effector cells” are cells of the immune system which can stimulate or trigger cytotoxicity, phagocytosis, antigen presentation, cytokine release. Such effector cells are, for example but not limited to, T cells, natural killer (NK) cells, granulocytes, monocytes, macrophages, dendritic cells, and antigen-presenting cells. Examples of suitable specificities for effector cells include but are not limited to CD2, CD3 and CD3 subunits such as CD3€, CD5, CD28 and other components of the T-cell receptor (TCR) for T cells; CD16 CD16A, CD25, CD38, CD44, CD56, CD69, CD94, CD335 (NKp46), CD336 (NKp44), CD337 (NKp30), NKp80, NKG2C and NKG2D, DNAM, NCRs for NK cells; CD18, CD64 and CD89 for granulocytes; CD18, CD32, CD64, CD89 and mannose receptor for monocytes and macrophages; CD64 and mannose receptor for dendritic cells; as well as CD35. In certain embodiments of the invention those specificities, i.e. cell surface molecules, of effector cells are suitable for mediating cell killing upon binding of a trispecific antigen-binding molecule to such cell surface molecule and, thereby, inducing cytolysis or apoptosis.

CD3 antigen is associated with the T-cell receptor complex on T-cells. In the case where specificity for an effector cell is CD3, the binding of the antigen-binding molecule according to the invention to CD3 triggers the cytotoxic activity of T-cells. By binding of the antigen-binding molecule to CD3 and to a target cell, e.g. tumor cell, cell lysis of the target cell may be induced.

The CD16A (FcyIIIA) antigen is a receptor expressed on the surface of NK cells. NK cells possess an inherent cytolytic activity and by binding of the antigen-binding molecule according to the invention to CD16 or CD16A the cytotoxic activity of NK cell towards the target can be triggered.

“Target” is the site on which the antigen epitope is located and to which the antigen-binding molecule should bind to. Examples of targets are cells, infectious agents such as viral or bacterial pathogens, for example dengue virus, herpes simplex, influenza virus, HIV, HCV or cells carrying autoimmune targets such as IL-2/IL2R, an autoimmune marker or an autoimmune antigen or tumor cells. In embodiments, wherein at least one of the antigen-binding sites has specificity for an effector cell, the target can be a tumor cell to which the effector cell should be redirected to induce or trigger the respective biological, e.g. immune, response.

Suitable specificities for tumor cells may be tumor antigens and cell surface antigens on the respective tumor cell, for example specific tumor markers. The term “tumor antigen” as used herein may comprise tumor associated antigen (TAA) and tumor specific antigen (TSA). A “tumor associated antigen” (TAA) as used herein refers to a protein which is present on tumor cells, and on normal cells during fetal life (once-fetal antigens), and after birth in selected organs, but at much lower concentration than on tumor cells. A TAA may also be present in the stroma in the vicinity of the tumor cell but expressed at lower amounts in the stroma elsewhere in the body. In contrast, the term “tumor specific antigen” (TSA) refers to a protein expressed by tumor cells. The term “cell surface antigen” refers to a molecule any antigen or fragment thereof capable of being recognized by an antibody on the surface of a cell.

Examples of specificities for tumor cells include but are not limited to CD19, CD20, CD26, CD29, CD30, CD33, CD52, CD200, CD267, EGFR, EGFR2, EGFR3, EGFRvIII, HER2, HER3, IGFR, IGF-1R, Ep-CAM, PLAP, Thomsen-Friedenreich (TF) antigen, TNFRSF17, gpA33, MUC-1 (mucin), IGFR, CD5, IL4-R alpha, IL13-R, Fc€RI, MHCI/peptide complexes and IgE.

Antigen-binding molecules according to the invention, wherein the tumor specificity is towards CD19 antigen may be used for immunotherapy of B-cell malignancies, because the CD19 antigen is expressed on virtually all B-lineage malignancies from lymphoblastic leukemia (ALL) to non-Hodgkin's lymphoma (NHL).

Antigen-binding molecules according to the invention wherein the tumor specificity is towards CD30 may be particularly useful in treating Hodgkin's disease and T-cell lymphomas.

For increasing serum-half life of the antigen-binding molecule according to the invention in the body, the antigen-binding molecule, if desired, may be fused to albumin, e.g. HSA, or pegylated, sialylated or glycosylated (see, for example, Stork et al., 2008, J. Biol. Chem., 283:7804-7812).

In some embodiments the trispecific antigen-binding molecule may comprise at least one antigen binding site, wherein the VH and VL domains of the VH/VL pair of the antigen binding site are non-covalently bonded with one another, i.e. the VH and VL domains of this VH/VL pair are not linked by a peptide linker or a peptide bond. In certain embodiments these non-covalently bonded VH and VL domains are located on different, i.e. a first and a second, polypeptide chains of a multimeric antigen-binding molecule. In other embodiments these non-covalently bonded VH and VL domains are located on the same polypeptide chain of a monomeric antigen-binding molecule, wherein at least another variable domain is arranged on the monomer in between each of these non-covalently bonded VH and VL domains. In some embodiments each of these non-covalently bonded VH and VL domains of this antigen binding site is bonded by a peptide linker or peptide bond to a VH or a VL domain of a second VH/VL pair of a juxtaposed antigen binding site. Preferably, such peptide linker to a VH or a VL domain of a VH/VL pair of a juxtaposed antigen binding site is short for preventing intramolecular folding between the juxtaposed domains and for forcing the association of the two non-covalently bonded VH and VL domains with each other. For example the peptide linker may comprise 12 or less amino acid residues, preferably 3 to 9 amino acid residues. Such a generation of at least one antigen binding site by two non-covalently bonded VH and VL domains is advantageous for the stability of the antigen-binding molecule, because it leads to a more compact antigen-binding molecule.

For example FIGS. 1 and 2 show trispecific antigen-binding molecule wherein the VH and VL domains of the central VH/VL pairs (illustrated in black) are non-covalently bonded with one another. In this example the non-covalently bonded VH and VL domains are located on different polypeptide chains. Each of these non-covalently bonded VH and VL domains of this antigen is bonded by a peptide linker L3 or L4 to a VH or a VL domain of a second VH/VL pair of a juxtaposed antigen binding site.

In further embodiments the trispecific antigen-binding molecule may comprise at least one first antigen binding site, wherein the VH and VL domains of the VH/VL pair of this first antigen binding site are non-covalently bonded with one another, i.e. the VH and VL domains of this VH/VL pair are not linked by a peptide linker or a peptide bond and the non-covalently bonded VH domain of this first antigen binding site is bonded by a peptide linker to a VH domain of a VH/VL pair of a second antigen binding site located juxtaposed to the first antigen-binding site and the non-covalently bonded VL domain of the first antigen binding site is bonded by a peptide linker to a VL domain of a VH/VL pair of the second antigen binding site located juxtaposed to the first antigen-binding site. In embodiments, where the antigen-binding molecule is a single-chain, i.e. monomeric, polypeptide, the VH and VL domains are arranged on the same polypeptide chain. In embodiments where the antigen-binding molecule is a multimeric, i.e. multi-chain, polypeptide, the VH domain of the first antigen binding site bonded by a peptide linker or peptide bond to a VH domain of the second antigen site are located on a first polypeptide and the VL domain of the first antigen binding site bonded by a peptide linker or peptide bond to a VL domain of a second antigen binding site are located on a second polypeptide. Preferably, the peptide linker is short, e.g. less than 12 amino acid residues, preferable 3 to 9 amino acid residues, for preventing intramolecular folding between the juxtaposed VH-VH and juxtaposed VL-VL domains, respectively, and forcing the association of the VH-VH domains with the VL-VL domains for forming the first and the second antigen binding sites. This VH-VH and VL-VL domain arrangement facilitates the correct folding of the trispecific antigen-binding molecule.

“Antigen-binding molecule” refers to a molecule of an immunoglobulin derivative with multivalent antigen-binding properties, preferably having at least four antigen-binding sites. The antigen-binding molecule can be a single-chain, i.e. monomeric, polypeptide or a multichain, i.e. multimeric polypeptide. Each polypeptide of the antigen-binding molecule may comprise antibody variable (Fv) domains linked with one another by a peptide linker or a peptide bond. Each antigen-binding site is formed by an antibody, i.e. immunoglobulin, variable heavy domain (VH) and an antibody variable light domain (VL) binding to the same antigen epitope. The antigen epitope may be on the same or different antigens. Preferably, the antigen-binding molecule according to the invention is devoid of immunoglobulin constant domains or fragments thereof.

The term “polypeptide” refers to a polymer of amino acid residues linked by amide bonds. The polypeptide is, preferably, a single chain fusion protein which is not branched. Within the polypeptide the antibody variable (Fv) domains are linked one after another. The polypeptide may have contiguous amino acid residues in addition N-terminal and/or C-terminal. For example, the polypeptide may contain a Tag sequence, preferably at the C-terminus which might be useful for the purification of the polypeptide. Example of a Tag sequence are a His-Tag, e.g. a His-Tag consisting of six His-residues, a FLAG™, e.g. a DYKDDDDK octapeptide (SEQ ID NO: 5) or STREP® II, e.g a WSHPQFEK octapeptide (SEQ ID NO: 6). For a multimeric antigen-binding molecule, preferably, different Tag sequences are used for different polypeptides.

Regarding the amino acid composition of the peptide linkers, peptides are selected that do not interfere with the association of the domains as well as do not interfere with the multimerization, e.g. dimerization, of multimeric molecules. For example, linkers which may comprise glycine and serine residues generally provide protease resistance. The amino acid sequence of the linkers can be optimized, for example, by phage-display methods to improve the antigen binding and production yield of the antigen-binding molecule. In an embodiment (G₂S)ₓ peptide linkers are used.

In some embodiments of the invention at least one, preferably all, antibody variable domains are fully human, humanized or chimeric domains. Humanized antibodies can be produced by well-established methods such as, for example CDR-grafting (see, for example, Antibody engineering: methods and protocols/edited by Benny K. C. Lo; Benny K. C. II Series: Methods in molecular biology (Totowa, N.J.). Thus, a skilled person is readily able to make a humanized or fully human version of antigen-binding molecule and variable domains from non-human, e.g. murine or non-primate, sources with the standard molecular biological techniques known in the art for reducing the immunogenicity and improving the efficiency of the antigen-binding molecule in a human immune system. In a preferred embodiment of the invention all antibody variable domains are humanized or fully human; most preferred, the antigen-binding molecule according to the invention is humanized or fully human. The term “Fully human” as used herein means that the amino acid sequences of the variable domains and the peptides linking the variable domains in the polypeptide originate or can be found in humans. In certain embodiments of the invention the variable domains may be human or humanized but not the peptides linking the antibody variable domains.

In some embodiments the present invention provides a multifunctional antigen-binding polypeptide multimer.

In some embodiments, the present invention provides an antigen-binding molecule of a trifunctional antigen-binding polypeptide multimer designed to target three different antigens or epitopes. Such a multimer may comprise a first polypeptide and a second polypeptide. Each of the two polypeptides is a single-chain fusion peptide having at least four antibody variable domains linked one after another from the N- to the C-terminus of each polypeptide. Each of the polypeptides may comprise two antibody variable domains linked by a short linker for preventing intramolecular pairing within the same polypeptide and a single-chain Fv unit having an antibody variable domain pair of the other two variable domains capable of intramolecularly forming an antigen binding site by the variable domain pair within the same polypeptide. The multimer is formed by non-covalent association between the two polypeptides, whereas the two antibody variable domains linked by a short linker of one polypeptide associated with the two corresponding antibody variable domains of the other polypeptide, thereby forming two additional antigen binding sites. Thus, this multimer may comprise at least four antigen binding sites and is at least tetravalent. In a particular aspect of the invention the multimer is a dimer, i.e. consists of two polypeptide chains.

For generating such a trispecific and tetravalent antigen-binding dimer the two polypeptides have to be of different antibody variable domain compositions, because with respect to at least one of the three specificities the respective antibody variable light domain and variable heavy domain have to be inserted into different polypeptides such that one of the polypeptides contains only the variable heavy domain and the other polypeptide contains only the variable light domain for this specificity. Thus, such a dimer according to the invention is heterodimeric, because it is composed of two different polypeptides.

Particular measures can be taken for enabling a correct association of the two different polypeptides which may comprise antibody variable domains for three different specificities and to prevent a wrong homodimerization between two identical polypeptides. For example, the inventors have obtained a correct heterodimerization between the two different, trispecific polypeptides by inserting two antibody variable heavy domains linked by a short linker in one polypeptide and inserting the two corresponding antibody variable light domains linked by a short linker into the other polypeptide. Surprisingly, only heterodimeric species of the trispecific antigen-binding polypeptide dimers have been formed.

Therefore, in an embodiment the invention provides
a trispecific antigen-binding molecule, wherein said molecule is a trispecific antigen-binding polypeptide dimer which may comprise a first polypeptide and a second polypeptide, each polypeptide having at least four antibody variable domains linked one after another, and
(a) the first polypeptide may comprise a first and a second antibody variable heavy domain (VH) linked with each other by a first linker preventing intramolecular pairing within the same polypeptide, for example of about 12 or less amino acid residues, and a single chain Fv antigen binding unit having a third antibody variable heavy domain (VH) linked by a second peptide linker with an antibody variable light domain (VL), said third antibody variable heavy domain (VH) and antibody variable light domain (VL) are capable to associate to a first antigen binding site, wherein the second antibody variable heavy domain (VH) is linked with the single chain Fv antigen binding unit by a third peptide linker; and
(b) the second polypeptide may comprise a first and a second antibody variable light domain (VL) linked with each other by a second peptide linker preventing intramolecular pairing within the same polypeptide, for example of about 12 or less amino acid residues, and a single chain Fv antigen binding unit having a third antibody variable domain (VL) linked by a second peptide linker with an antibody variable heavy domain (VH), said third antibody variable light domain (VL) and antibody variable heavy domain (VH) are capable to associate to a second antigen binding site, wherein the second antibody variable light domain (VL) is linked with the single chain Fv antigen binding unit by a third peptide linker; and
(c) the first and the second antibody variable heavy domain (VH) of the first polypeptide associated with the first and the second antibody variable light domain (VL) of the second polymer to two additional, i.e. a third and fourth, antigen binding sites, whereas in a preferred embodiment the first antibody variable heavy domain (VH) of the first polypeptide associates with the second antibody light chain region (VL) of the second polypeptide to a third antigen binding site and the second antibody variable heavy domain (VH) of the first polypeptide associates with the first antibody variable light domain (VL) of the second polypeptide to a fourth antigen binding site.

A trispecific antigen-binding polypeptide dimer is formed, when two of said four antigen binding sites are specific for the same antigen.

Such a trispecific dimer recognizes three different specificities, and can target, for example, two different antigens or epitopes on a target cell and with the third functionality, i.e. specificity, bind, for example, to an immune effector cell such as, for example, a T- or a NK-cell.

The trispecific dimer according to the invention can be utilized in different ways.

For example, the antibody variable domains may be arranged within a polypeptide such that the two antibody variable domains associating with the two corresponding antibody variable domains of the other polypeptide may be positioned, for example, at the N-terminus or the C-terminus of the polypeptide. These two antibody variable domains may have the same specificity or distinct specificities. For example, both may be specific for the same immune effector cell or have distinct specificities for two antigens on a tumor cell.

Further, the two antibody variable domains forming the single-chain Fv unit may be, for example, in the order VH-VL or VL-VH in the direction from the N- to the C-terminus of the polypeptide. The single-chain Fv units of the two dimerized polypeptides may have the same or different specificities. For example, if the two antibody variable domains associating with the two corresponding antibody variable domains of the other polypeptide have the same specificity, the single-chain Fv units of the two polypeptides have different specificities for achieving a trispecific dimer.

Thus, the at least four antibody variable domains may be arranged, for example, such that the two antibody variable domains associating with the two corresponding antibody variable domains of the other polypeptide are specific for an immune effector cell and the single-chain Fv units of the two polypeptides have specificities for two distinct tumor antigens or the two antibody variable domains associating with the two corresponding antibody variable domains of the other polypeptide are specific for distinct tumor antigens and both single-chain Fv units of the two polypeptides have the same specificity for an immune effector cell.

The antigen-binding polypeptide is a “dimer” which term refers to a complex of a first and a second polypeptide monomer. In one aspect the antigen-binding polypeptide dimer is a “heterodimer” which term means that the antigen-binding polypeptide is composed of two different polypeptide monomers that are encoded by two distinct polynucleotides.

Preferably, in the antigen-binding dimer the first and the second polypeptides are non-covalently associated with each other, in particular with the proviso that there is no covalent bound between the first and second polypeptide. However, if desired, the two polypeptides may be additionally stabilized by at least one covalent linkage, e.g. by a disulfide bridge between cysteine residues of different polypeptides.

The length of the linkers influences the flexibility of the antigen-binding polypeptide dimer. The desired flexibility of the antigen-binding polypeptide dimer depends on the target antigen density and the accessibility of the target antigen, i.e. epitopes. Longer linkers provide a more flexible antigen-binding polypeptide dimer with more agile antigen-binding sites. The effect of linker length on the formation of dimeric antigen-binding polypeptides is described, for example, in Todorovska et al., 2001 Journal of Immunological Methods 248:47-66; Perisic et al., 1994 Structure 2:1217-1226; Le Gall et al., 2004, Protein Engineering 17:357-366 and WO 94/13804.

According to the invention it is preferred that the length of the first peptide linker of the first and second antibody variable heavy domains of the first polypeptide and the first and second antibody variable light domains of the second polypeptide is such that the domains of the first polypeptide can associate intermolecularly with the domains of the second polypeptide to form the dimeric antigen-binding polypeptide. Such linkers are “short”, i.e. consist of 0, 1, 2, 3, 4, 5, 6, 7, 8, 9, 10, 11 or about 12 amino acid residues. In the case of 0 amino acid residues the linker is a peptide bond. Such short linkers favor the correct dimerization of the first with the second polypeptide by binding and forming antigen-binding sites between antibody variable light domains and antibody variable heavy domains of different polypeptides. Shortening the linker to about 12 or less amino acid residues generally prevents adjacent domains of the same polypeptide chain from interacting with each other. In an embodiment of the invention these linkers consist of about 3 to about 10, for example 7 contiguous amino acid residues. Besides, it is in principle possible that two polypeptides having a linker with more than 12 amino acid residues between the variable antibody domains correctly dimerize with one another (see for example Le Gall et al., 2004, Protein Engineering 17:357-366).

For the single-chain Fv units of the polypeptides the second peptide linker is long and flexible (in general consisting of about 12 or more amino acid residues) for folding intramolecularly head-to-tail and forming the single-chain antigen-binding (scFv) unit. Additional amino acid residues provide extra flexibility. For example this linker between the VH and VL or VL and VH of the single-chain Fv unit in the polypeptide may consist of about 12 to about 35, in particular from 15 to 25 contiguous amino acid residues.

The third peptide linker of the polypeptide for linking the single-chain Fv unit with the other two antibody variable domains which associate with the corresponding variable domains of the other polypeptide may be, for example, from 5 to 30, preferably at least 6, 7, 8, 9, 10, 11, or 12 contiguous amino acid residues.

In an embodiment of the invention the trispecific antigen-binding polypeptide dimer is bispecific for two distinct antigens on a tumor cell and additionally specific for an effector cell, in particular a T cell or a NK cell. Suitable specificities for tumor cells may be tumor antigens and cell surface antigens on the respective tumor cell, for example specific tumor markers. Such a trispecific antigen-binding dimer binds bifunctionally to a tumor cell and to the immune effector cell thereby triggering the cytotoxic response induced by the T cell or the NK cell.

The antigen-binding molecule according to any one of the embodiments described here previously may be produced by expressing polynucleotides encoding the individual polypeptide chains which form the antigen-binding molecule. Therefore, a further embodiment of the invention are polynucleotides, e.g. DNA or RNA, encoding the polypeptide chains of the antigen-binding molecule as described herein above.

The polynucleotides may be constructed by methods known to the skilled person, e.g. by combining the genes encoding the antibody variable domains either separated by peptide linkers or directly linked by a peptide bound of the polypeptides, into a genetic construct operably linked to a suitable promoter, and optionally a suitable transcription terminator, and expressing it in bacteria or other appropriate expression system such as, for example CHO cells. Depending on the vector system and host utilized, any number of suitable transcription and translation elements, including constitutive and inducible promoters, may be used. The promoter is selected such that it drives the expression of the polynucleotides in the respective host cell.

The polynucleotides may be inserted into vectors, preferably expression vectors, which represent a further embodiment of the invention. These recombinant vectors can be constructed according to methods well known to the person skilled in the art.

A variety of expression vector/host systems may be utilized to contain and express the polynucleotides encoding the polypeptide chains of the present invention. Examples for expression vectors for expression in E. coli is pSKK (LeGall et al., J Immunol Methods. (2004) 285(1):111-27) or pcDNA5 (Invitrogen) for the expression in mammal cells.

Thus, the antigen-binding molecule as described herein may be produced by introducing a vector encoding the polypeptide chains as described above into a host cell and culturing said host cell under conditions whereby the polypeptide chains are expressed, may be isolated and, optionally, further purified.

In a further embodiment of the invention compositions which may comprise an antigen-binding molecular polynucleotides as described herein above and at least one further component are provided.

The invention further provides a method wherein the antigen-binding molecule as described herein above is administered in an effective dose to a subject, e.g., patient, for the treatment of cancer (e.g. non-Hodgkin's lymphoma; chronic lymphocytic leukaemia). The antigen-binding molecule can be used as a medicament.

A skilled person will readily be able without undue burden to construct and obtain the antigen-binding molecule described herein by utilizing established techniques and standard methods known in the art, see for example Sambrook, Molecular Cloning A Laboratory Manual, Cold Spring Harbor Laboratory (1989) N.Y.; The Protein Protocols Handbook, edited by John M. Walker, Humana Press Inc. (2002); or Antibody engineering: methods and protocols/edited by Benny K. C. Lo; Benny K. C. II Series: Methods in molecular biology (Totowa, N.J.)).

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### Example 1

### DNA Constructs:

The plasmid DNA encoding the polypeptide chains are generated by DNA engineering or by gene synthesis and sequencing. The expression constructs for transient or stable transfection of mammalian cells are based on the eukaryotic expression vector pCDNA5/FRT (Life Technologies) and comprise the product gene of interest under the control of a viral or ubiquitous promoter, as well as a Hygromycin resistance cassette as a selection marker. For purification and analytics, the product chains are expressed with His-tag, FLAG-tag or StrepII-tag.

### Cell Lines and Cell Cultivation:

Flp-In CHO cells (Life Technologies), a derivative of CHO-K1 Chinese Hamster ovary cells (ATCC, CCL-61) (Kao and Puck, 1968), are cultured in Ham's F-12 Nutrient Mix supplemented with L-Glutamine, 10% FCS and 100 μg/ml Zeocin. Adherent cells are detached with 0.25% Trypsin-EDTA and subcultured according to standard cell culture protocols.

For adaptation to growth in suspension, cells are detached from tissue culture flasks and placed in serum-free medium for subsequent incubation in shake flasks (Corning) at 37° C., 5% CO₂ and 120 rpm. The standard medium for the culture of suspension-adapted Flp-In CHO cells is HYCLONE™ CDM4 CHO (Thermo Scientific) supplemented with L-Glutamine (Life Technologies), HT Supplement (Life Technologies), Penicillin/Streptomycin (Life Technologies) and 100 μg/ml Zeocin (Life Technologies). Suspension-adapted cells are subcultivated every 2-3 days with seeding densities of 2E+6 to 3E+6 cells/ml. The cell concentration and viability is determined in all cultures using the trypan blue exclusion method. Cells are cryopreserved in medium with 10% DMSO and tested negative for Mycoplasma using MycoAlert Mycoplasma detection Kit (Lonza).

### Generation of Stably Transfected Cell Pools:

Recombinant Flp-In CHO cell lines stably expressing tri-specific candidate antibodies, are generated by transfection of suspension-adapted cells. For this, cells are placed in standard medium without Zeocin one day prior to co-transfection with expression plasmids (2.5 μg) encoding the protein of interest (pcDNA5/FRT) and the Flp recombinase (pOG44, Life Technologies) using Polyethylenimine (PEI). In brief, vector DNA and transfection reagent are mixed at a DNA:PEI mass ratio of 1:3 in a total of 100 μL OPTI-MEM™ I medium (Life Technologies) and incubated for 10 minutes before addition to 2E+6 Flp-In CHO cells suspended in 1 ml CHO-S-SFMII medium (Life Technologies). Following 24 h incubation, selection for stably transfected cells is started by addition of 500 μg/mL Hygromycin B subsequent to diluting cultures to a density of 0.1E+6 viable cells/mL in CHO-S-SFMII medium and seeding in T75 culture flasks. Flp recombinase mediates the insertion of the Flp-In expression construct into the genome at the integrated FRT site through site-specific DNA recombination (O'Gorman et al 1991). During selection viable cell densities are measured twice a week, and cells are centrifuged and resuspended in fresh selection medium at a maximal density of 0.1E+6 viable cells/mL. Cell pools stably expressing recombinant protein products are recovered after approximately 3 weeks of selection at which point cells are transferred to standard culture medium in shake flasks. Expression of recombinant secreted proteins is confirmed by protein gel electrophoresis of cell culture supernatants using Criterion Stain-Free (Bio-Rad) technology. Stable cell pools are cryopreserved in medium containing 50% ProFreeze (Lonza) and 7.5% DMSO.

### Production of Recombinant Protein in Fed-Batch CHO Cell Suspension Cultures:

Recombinant proteins are produced in 10-day fed-batch cultures of stably transfected CHO cell lines by secretion into the cell culture supernatant. For this, cell pools stably expressing the product of interest are seeded at starting densities of 6E+5 cells/mL in standard culture medium in polycarbonate Erlenmeyer flasks with gas permeable caps (Corning) and incubated at 37° C. and 5% CO2 with agitation at 140 rpm. During fed-batch culture, media is supplemented with 40 mL/L ActiCHO Feed A (PAA) and 4 mL/L ActiCHO Feed B (PAA) on day 0 (starting day), and with double amounts on day 3, 5, and 7. Cell culture supernatants are harvested after 10 days at culture viabilities of typically >75%. Samples are collected from the production cultures every other day prior to feeding and cell density and viability is assessed. On the day of harvest, cell culture supernatants are cleared by centrifugation and vacuum filtration (0.22 μm) using Millipore Express PLUS Membrane Filters (Millipore) before further use.

### Determination of Expression Titer:

Protein expression titers and product integrity in cell culture supernatants (CCS) are analysed by SDS-PAGE using the Criterion Stain-Free gel imaging system (Bio-Rad) on days 7 and 10 (before and after 0.22 μm filtration). Product titers are determined semi-quantitatively by comparison with a reference protein of known concentration.

### Purification of Trispecific Antigen-Binding Polypeptides:

His-tagged products are purified from CHO cell culture supernatants in a two-step procedure comprising Ni-NTA- and preparative size-exclusion chromatography. First, supernatants are cleared by vacuum filtration (0.22 μm) and adjusted to 5 mM imidazole before loading onto HISTRAP™ FF chromatography column (GE Healthcare) equilibrated in IMAC Buffer A at a flow rate of 5 mL/min. Columns are subsequently washed with 5 CV IMAC Buffer A and 10 CV of a mixture of IMAC Buffer A and IMAC Buffer B (7%). His-tagged products are then eluted by sequential washing with 10 CV 30% IMAC Buffer B and 5 CV 100% IMAC Buffer B at the same flow rate. 2.5 mL eluate fractions are collected and protein content and purity is assessed by subjecting each fraction to one-dimensional SDS-PAGE followed by visualization of protein using Criterion Stain-Free technology (Bio-Rad). Product containing fractions are pooled and concentrated by ultrafiltration. Subsequently, concentrated samples are purified by gel filtration using a HiLoad 26/600 Superdex 200 pg (GE Healthcare) column and eluted in SEC Buffer (20 mM Tris-HCl, 100 mM NaCl, pH 7.5) at 2.5 mL/min. Fractions containing the purified product, as determined by comparison of elution volumes with column retention of molecular weight marker proteins (GE Healthcare), are collected and pooled. After a final buffer exchange (10 mM sodium acetate, pH 5.0) using PD-10 desalting columns (GE Healthcare) samples are concentrated to 1.0-1.5 mg/mL by ultrafiltration as described above. Purity and homogeneity (typically >90%) of final samples are assessed by Criterion Stain-Free gel visualization of proteins after reducing and non-reducing SDS-PAGE as described above, in selected cases followed by immunoblotting with specific antibodies and by analytical SEC, respectively. Purified proteins are stored as aliquots at −80° C. until further use.

### Examples 2 CD3×CD19×CD30 Trispecific Molecules

Antigen-binding polypeptide dimers containing CD3-, CD19- and CD30-antibody variable binding domains originating from the antibodies OKT3, HD37 and HRS3, respectively are produced according to Example 1:

Trispec 1:
(SEQ ID NO: 1)
VH(CD3)-(G₂S)₂-VH(CD3)-(G₂S)₃-VH(CD30)-(G₂S)₅VL
(CD30)-His₆
(SEQ ID NO: 2)
VL(CD3)-(G₂S)₂-VL(CD3)-(G₂S)₃-VH(CD19)-(G₂S)₅VL
(CD19)-FLAG
Trispec 2:
(SEQ ID NO: 3)
VH(CD30)-(G₂S)₂-VH(CD19)-(G₂S)₂-VH(CD3)-(G₂S)₅-VL
(CD3)-His₆
(SEQ ID NO: 4)
VL(CD19)-(G₂S)₂-VL(CD30)-(G₂S)₂-VH(CD3)-(G₂S)₅-VL
(CD3)-FLAG
Linker 1 =
SEQ ID NO: 7
(G₂S)₂,
Linker 2 =
(SEQ ID NO: 8)
(G₂S)₅,
Linker 3 =
(SEQ ID NO: 9)
(G₂S)₃

Immunoprecipitation of Trispec 1 and Trispec 2 show that only heterodimeric species of the antigen-binding polypeptide dimer are detected. Trispec 1 and Trispec 2 exhibit excellent stability at 40° C. after 7 days and at pH 3.5 after 1 h.

### Example: Assessment of Cytotoxic Activity Mediated by Trispecific Antibodies

### Study Procedures

Isolation of PBMC from buffy coats and enrichment of T cells:
PBMCs are isolated from buffy coats by density gradient centrifugation. T cells are enriched from the PBMC population using the EasySep™ Human T Cell Enrichment Kit for the immunomagnetic isolation of untouched human T cells and the Big Easy EasySep™ Magnet according to the manufacturer's instructions.

### FACS-Based Cytotoxicity Assay:

T cells that are used as effector cells are characterized by flow cytometry as described.

Target cells (MEC-1: DSMZ, cat.: ACC 497; NALM-6: DSMZ, cat.: ACC 128) are cultured under standard conditions as described below. For the cytotoxicity assay target cells are harvested, washed twice with RPMI 1640 medium without FCS, and resuspended in diluent C provided in the PKH67 Green Fluorescent Cell Linker Mini Kit to a density of 2×10⁷/mL. The cell suspension is then mixed with the equal volume of a double-concentrated PKH67-labeling solution (e.g. 1 μL PKH67 in 250 μL diluent C) and incubated according to the manufacturer's instructions. The staining reaction is stopped. After washing the labeled target cells with complete RPMI medium, cells are counted and resuspended to a density of 2×10⁵/mL in complete RPMI medium.

2×10⁴ target cells are then seeded together with T cells at and the indicated antibodies in individual wells. Spontaneous cell death and killing of targets by effectors in the absence of antibodies are determined.

After incubation, cultures are washed once with FACS buffer and then resuspended in 150 μL FACS buffer supplemented with 2 μg/mL PI. The absolute amount of living target cells that are characterized by a positive green PKH67 staining but are negative for the PI staining are measured using a Beckman-Coulter FC500 MPL flow cytometer (Beckman-Coulter) or a Millipore Guava EasyCyte flow cytometer (Merck Millipore).

Based on the measured remaining living target cells, the percentage of specific cell lysis is calculated according to the following formula: [1-(number of living targets₍ₛₐₘₚₗₑ₎)/(number of living targets₍ₛₚₒₙₜₐₙₑₒᵤₛ₎)]×100%. Sigmoidal dose response curves and EC₅₀ values are calculated by non-linear regression/4-parameter logistic fit using the GraphPad Prism software (GraphPad Prism version 6.00 for Windows, GraphPad Software, La Jolla Calif. USA).

### Statistical Analysis

The lysis values obtained for a given antibody concentration are determined and analysed by sigmoidal dose-response/4 parameter logistic fit analysis using the Prism software (GraphPad Prism version 6.00 for Windows, GraphPad Software, La Jolla Calif. USA) and used to calculate EC₅₀ values, and mean and SD of replicates of percentage lysis.

### Results:

Trispec 1 and Trispec 2 exhibit higher cytotoxic potency on double-positive cell lines (CD19⁺ and CD30⁺) when compared to the respective single-positive cell lines.

The invention is further described by the following numbered paragraphs:

1. A trispecific antigen-binding molecule, wherein the antigen-binding molecule is at least tetravalent and comprises an antigen-binding site having specificity against a first antigen epitope, an antigen-binding site having specificity against a second antigen epitope and two antigen-binding sites having specificity against a third antigen epitope.

2. The trispecific antigen-binding molecule according to paragraph 1, wherein each of the antigen-binding sites consists of a VH/VL pair, wherein the VH and the VL domains of a first VH/VL pair are non-covalently bonded with one another and each of said non-covalently bonded VH and VL domains are bonded to another VH or VL domain of a second VH/VL pair located juxtaposed to the first VH/VL pair by a peptide linker or a peptide bond.

3. The trispecific antigen-binding molecule according to paragraph 2, wherein the VH domain of the VH/VL pair is bonded by a peptide linker or a peptide bond to a VH domain of the VH/VL pair and the VL domain of the first VH/VL pair is bonded by a peptide linker or a peptide bond to a VL domain of the second VH/VL pair.

4. The trispecific antigen-binding molecule according to anyone of paragraphs 1 to 3, wherein said molecule is an antigen-binding polypeptide dimer comprising a first polypeptide and a second polypeptide, each polypeptide has at least four antibody variable domains linked one after another, wherein
(a) the first polypeptide comprises a first and a second antibody variable domains linked with each other by a linker of about 12 or less amino acid residues and a single chain Fv antigen binding unit having an antibody variable heavy domain (VH) linked with an antibody variable light domain (VL), said antibody variable heavy domain (VH) and antibody variable light domain (VL) are capable to associate to a first antigen binding site, wherein the first or second antibody variable domain is linked with the single chain Fv antigen binding unit by a peptide linker;
(b) the second polypeptide comprises a first and a second antibody variable domain linked with each other by a linker of about 12 or less amino acid residues, wherein the first and second antibody variable domains and a single chain Fv antigen binding unit having an antibody variable light domain (VL) linked with an antibody variable heavy domain (VH), said antibody variable light domain (VL) and antibody variable heavy domain (VH) are capable to associate to a second antigen binding site and the first or the second antibody variable domain is linked with the single chain Fv antigen binding unit by a peptide linker;
(c) the first antibody variable domain of the first polypeptide associates with the second antibody variable domain of the second polypeptide to a third antigen binding site;
(d) the second antibody variable domain of the first polypeptide associates with the first antibody variable domain of the second polypeptide to a fourth antigen binding site; and
(e) two of said four antigen binding sites are specific for the same antigen.

5. The trispecific antigen-binding molecule, wherein the said molecule is an antigen-binding polypeptide dimer according to paragraph 4, wherein
(a) the first polypeptide comprises a first and a second antibody variable heavy domain (VH) linked with each other by a linker of about 12 or less amino acid residues and a single chain Fv antigen binding unit having a third antibody variable heavy domain (VH) linked with an antibody variable light domain (VL), said third antibody variable heavy domain (VH) and antibody variable light domain (VL) are capable to associate to a first antigen binding site, wherein the first or second antibody variable heavy domain (VH) is linked with the single chain Fv antigen binding unit by a peptide linker;
(b) the second polypeptide comprises a first and a second antibody variable light domain (VL) linked with each other by a linker of about 12 or less amino acid residues and a single chain Fv antigen binding unit having a third antibody variable light domain (VL) linked with an antibody variable heavy domain (VH), said third antibody variable light domain (VL) and antibody variable heavy domain (VH) are capable to associate to a domain (VL) is linked with the single chain Fv antigen binding unit by a peptide linker;
(c) the first antibody variable heavy domain (VH) of the first polypeptide associates with the second antibody variable light domain (VL) of the second polypeptide to a third antigen binding site;
(d) the second antibody variable heavy domain (VH) of the first polypeptide associates with the first antibody variable light domain (VL) of the second polypeptide to a fourth antigen binding site; and
(e) two of said four antigen binding sites are specific for the same antigen.

6. The antigen-binding molecule according to paragraph 4 or 5, wherein the first and the second variable domains of the first polypeptide and the first and the second variable domains of the second polypeptide are linked by a linker having 3 to 9 amino acid residues.

7. The antigen-binding molecule according to anyone of paragraphs 4 to 6, wherein the second variable domain and the single chain Fv unit of the first polypeptide and the second variable domain and the single chain Fv unit of the second polypeptide are linked with a linker having 2 to 35 amino acid residues.

8. The antigen-binding molecule according to anyone of paragraphs 4 to 7, wherein the variable heavy domain and the variable light domain of the single chain Fv unit of the first polypeptide and the variable light domain and the variable heavy domain of the single chain Fv unit of the second polypeptide are linked with a linker having 12 or more amino acid residues.

9. The antigen-binding molecule according to anyone of paragraphs 4 to 8, wherein the first and second antigen binding sites of the two single chain Fv units are specific for the same antigen.

10. The antigen-binding molecule according to anyone of paragraphs 4 to 9, wherein the third and the fourth antigen binding sites formed by association between the first and the second polypeptide are specific for the same antigen.

10. The antigen-binding molecule according to anyone of paragraphs 1 to 9, wherein the two antigen binding sites specific for the same antigen are specific for an antigen presented on a T-cell or natural killing (NK) cell.

11. The antigen-binding molecule according to paragraph 10, wherein the antigen is CD3, CD16 or CD16A.

12. The antigen-binding molecule according to paragraph 10 or 11, wherein the other two binding sites are specific for two different antigens on the same cell.

13. The antigen-binding molecule according to paragraph 12, wherein the cell is a tumor cell.

14. The antigen-binding molecule according to paragraph 13, wherein the two different antigens are selected from the group consisting of CD19, CD20, CD26, CD29, CD30 CD33, CD200, CD267, EGFR, EGFRvIII, HER2, HER3, IGFR, IGF-1R, Ep-CAM, PLAP, Thomsen-Friedenreich (TF) antigen, MUC-1 (mucin), CD5, IL4-R alpha, IL13-R, Fc€RI and IgE, gpA33, MHCI/peptide complexes.

15. The antigen-binding molecule according to anyone of paragraphs 1 to 9, wherein a first antigen binding site and a second antigen binding site are specific for two different antigen epitopes presented on a natural killing (NK) cell.

16. A vector encoding an antigen-binding molecule according to anyone of paragraphs 1 to 15.

17. A host cell transformed by an vector according to paragraph 16.

18. An antigen-binding molecule according to paragraphs 13 or 14 for use in tumor therapy.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A trispecific antigen-binding molecule comprising two polypeptides, each having variable antibody domains linked one after another, but being devoid of immunoglobulin constant domains, wherein
(a) a first polypeptide comprises a first and a second antibody variable heavy domain (VH) linked with each other by a linker of 12 or less amino acid residues and a single chain Fv antigen binding unit having a third antibody variable heavy domain (VH) linked with an antibody variable light domain (VL), wherein said third antibody variable heavy domain (VH) and antibody variable light domain (VL) are capable to associate to form a first antigen binding site specific for a first antigen, wherein the first or second antibody variable heavy domain (VH) is linked with the single chain Fv antigen binding unit by a peptide linker;
(b) a second polypeptide comprises a first and a second antibody variable light domain (VL) linked with each other by a linker of 12 or less amino acid residues and a single chain Fv antigen binding unit having a third antibody variable light domain (VL) linked by a peptide linker with an antibody variable heavy domain (VH), wherein said third antibody variable light domain (VL) and antibody variable heavy domain (VH) are capable to associate to form a second antigen binding site specific for a second antigen, wherein the first or second antibody variable light domain (VL) is linked with the single chain Fv antigen binding unit by a peptide linker;
(c) the first and second antibody variable heavy domains (VH) of the first polypeptide associate with the first and second antibody variable light domains (VL) of the second polypeptide to form two antigen binding sites specific for a third antigen; and
(d) each of the first and the second polypeptides comprises a Tag sequence and the Tag sequences are different from each other.

2. The antigen-binding molecule according to claim 1, wherein the Tag sequences are selected from a His-tag consisting of six His residues and SEQ ID NOS: 5 or 6.

3. The antigen-binding molecule according to claim 1, wherein the first and the second variable domains of the first polypeptide and the first and the second variable domains of the second polypeptide are linked by a linker having 3 to 9 amino acid residues.

4. The antigen-binding molecule according to claim 1, wherein in each polypeptide the second variable domain and the single chain Fv unit are linked with a linker having 2 to 35 amino acid residues.

5. The antigen-binding molecule according to claim 1, wherein the variable heavy domain and the variable light domain of each single chain Fv unit are linked with a linker having 12 or more amino acid residues.

6. The antigen-binding molecule according to claim 1, wherein the third and the fourth antigen binding sites formed by association between the first and the second polypeptide are specific for the same antigen.

7. The antigen-binding molecule according to claim 6, wherein the antigen-binding molecule specifically binds to an immune effector cell.

8. The antigen-binding molecule according to claim 7, wherein the antigen-binding molecule specifically binds to a T-cell or a NK-cell.

9. The antigen-binding molecule according to claim 8, wherein the antigen-binding molecule specifically binds to a NK-cell.

10. The antigen-binding molecule according to claim 6, wherein the antigen-binding molecule specifically binds to an immune effector cell and a tumor cell.

11. The antigen-binding molecule according to claim 10, wherein the immune effector cell is a NK cell.

12. A vector encoding an antigen-binding molecule according to claim 1.

13. A host cell transformed by a vector according to claim 12.


SEQUENCE LISTING (9 sequences)

SEQ ID NO: 1 - protein, 509 aa, artificial sequence
  polypeptide chain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGSGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGSGGSGGSQVQLQQSGAELARPGASVKMSCKASGYTFTTYTIHWVRQRPGHDLEWIGYINPSSGYSDYNQNFKGKTTLTADKSSNTAYMQLNSLTSEDSAVYYCARRADYGNYEYTWFAYWGQGTTVTVSSGGSGGSGGSGGSGGSDIVMTQSPKFMSTSVGDRVTVTCKASQNVGTNVAWFQQKPGQSPKVLIYSASYRYSGVPDRFTGSGSGTDFTLTISNVQSEDLAEYFCQQYHTYPLTFGGGTKLEINAAAGSHHHHHH

SEQ ID NO: 2 - protein, 490 aa, artificial sequence
  polypeptide chain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGSGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGSGGSGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVSSGGSGGSGGSGGSGGSDILLTQTPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEINAAAGSDYKDDDDK

SEQ ID NO: 3 - protein, 513 aa, artificial sequence
  polypeptide chain
QVQLQQSGAELARPGASVKMSCKASGYTFTTYTIHWVRQRPGHDLEWIGYINPSSGYSDYNQNFKGKTTLTADKSSNTAYMQLNSLTSEDSAVYYCARRADYGNYEYTWFAYWGQGTTVTVSSGGSGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVSSGGSGGSGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGSGGSGGSGGSGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINAAAGSHHHHHH

SEQ ID NO: 4 - protein, 486 aa, artificial sequence
  polypeptide chain
DILLTQTPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEINGGSGGSDIVMTQSPKFMSTSVGDRVTVTCKASQNVGTNVAWFQQKPGQSPKVLIYSASYRYSGVPDRFTGSGSGTDFTLTISNVQSEDLAEYFCQQYHTYPLTFGGGTKLEINGGSGGSGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGSGGSGGSGGSGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINAAAGSWSHPQFEK

SEQ ID NO: 5 - protein, 8 aa, artificial sequence
  Tag-sequence
DYKDDDDK

SEQ ID NO: 6 - protein, 8 aa, artificial sequence
  Tag-sequence
WSHPQFEK

SEQ ID NO: 7 - protein, 6 aa, artificial sequence
  Linker
EESEES

SEQ ID NO: 8 - protein, 15 aa, artificial sequence
  Linker
EESEESEESEESEES

SEQ ID NO: 9 - protein, 9 aa, artificial sequence
  Linker
EESEESEES